(19) United States
(12) Patent Application Publication
(10) Pub. No.: US 2017/0098049 A1
(43) Pub. Date: Apr. 6, 2017

(54) SYSTEM AND METHOD FOR TRACKING MEDICAL DEVICE INVENTORY

(71) Applicant: Spinal Generations, LLC, Mokena, IL (US)

(72) Inventor: Patrick J. Sweeney, Flossmoor, IL (US)

(21) Appl. No.: 15/282,289

(22) Filed: Sep. 30, 2016

Related U.S. Application Data
(60) Provisional application No. 62/236,724, filed on Oct. 2, 2015.

(51) Int. Cl. G06F 19/00; A61B 90/98; A61B 90/00; G06Q 10/08; A61B 34/00

(52) U.S. Cl. CPC G06F 19/3406; G06F 19/328; G06Q 10/087; A61B 34/25; A61B 90/361; A61B 90/37; A61B 90/98; A61B 2090/0805; A61B 2090/367

(57) ABSTRACT

A virtual tray system includes a tray inventory system configured to track a current inventory of a tray, with one or more details associated with each item in the tray. The inventory includes a plurality of items, such as medical devices, instruments, tools, or implants. The virtual tray system further includes a database configured to store the current inventory of the tray and a user interface system configured to generate a visual representation of the inventory of the tray for display on a user interface of a user device. The virtual tray system further includes a communications system configured to receive an input relating to a change in status of at least one of the items in the tray. Based on the input, the tray inventory system is configured to update the current inventory of the tray and update the visual representation of the tray based on the input.

### CROSS-REFERENCE TO RELATED APPLICATIONS

This application claims the benefit of and priority to U.S. Provisional Application No. 62/236,724, filed Oct. 2, 2015, which is incorporated herein by reference in its entirety.

### BACKGROUND

The present invention relates to the tracking of inventory (i.e., implants, tools, and other objects that may be used in a surgical procedure) in the medical device field. More particularly, the present invention relates to the tracking of inventory after the inventory has left the manufacturer (e.g., as part of a bulk package in a tray) and has been put into use at a medical facility. Currently, tracking surgical or medical device inventory is difficult. Further, there is a need to comply with government regulations regarding unique device identifying codes for devices that are bulk packaged in trays intended for repetitive steam sterilization prior to implantation or use.

In current systems for providing implants and tools during an operation, a large choice of devices may be provided to the operating area for the surgeon (or other personnel), allowing for the individualization of choice by the surgeon for the patient. In other words, the surgeon is provided with many options during an operation in order to make the best decision for the patient and operation. However, after the devices (and other medical items) has left the manufacturer and before the devices reaches the operating area, middlemen such as distributors, sales representatives, and hospital central sterilization departments may be involved. The system may not foster reliable tracking and product control policies for these middlemen, often leading to shrinkage, wastage, loss, and increased expense for the manufacturer and/or medical facility.

Current FDA/EU regulations require more specific identification of implants to allow better tracking for patient safety and for outcome evaluation. In a tray with hundreds of implants intended for steam sterilization repetitively, this may become obtrusive and unworkable in current systems.

### SUMMARY

One embodiment of the present disclosure relates to a virtual tray system. The virtual tray system includes a tray inventory system configured to track a current inventory of a tray, the inventory including a plurality of items, and further configured to associate one or more details specified for each item in the tray. The virtual tray system further includes a database configured to store the current inventory of the tray. The virtual tray system further includes a user interface system configured to generate a visual representation of the inventory of the tray for display on a user interface of a user device. The virtual tray system further includes a communications system configured to receive an input relating to a change in status of at least one of the plurality of items in the tray. Based on the input, the tray inventory system is configured to update the current inventory of the tray in the database, and the user interface system is configured to update the visual representation of the tray based on the input.

Another embodiment of the present disclosure relates to a method. The method includes receiving from a manufacturer, by a virtual tray system, tray inventory information for a tray, wherein the inventory information includes one or more details for a plurality of items in the tray. The method further includes storing, by the virtual tray system, the tray inventory information in a database. The method further includes receiving, by the virtual tray system, updated tray inventory information from a distributor that received the tray from the manufacturer and updating, by the virtual tray system, the tray inventory information in the database. The method further includes receiving, by the virtual tray system, updated tray inventory information from a medical facility that received the tray from the distributor and updating, by the virtual tray system, the tray inventory information in the database. The method further includes receiving an input relating to a change in status of at least one of the plurality of items in the tray during a medical procedure and updating, by the virtual tray system, the tray inventory information in the database.

Another embodiment of the present disclosure relates to a method. The method includes receiving, at a virtual tray system from a monitoring system, an indication of the use of an item in a tray. The method further includes updating, by the virtual tray system, an inventory of the tray based on the use of the item. The method further includes updating, by the virtual tray system, a database configured to store the current inventory of the tray. The method further includes updating, by the virtual tray system, a visual representation of the tray for display on a user device.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is an illustration of a virtual tray, associated with an actual tray, that is provided for the tracking and identification of medical devices and related items, according to an exemplary embodiment.

FIG. 2 is an illustration of an operating room in which the systems and methods described herein may be implemented, according to an exemplary embodiment.

FIG. 3 is a block diagram of a virtual tray system for maintaining a virtual tray associated with an actual tray, according to an exemplary embodiment.

FIG. 4 is an illustration of the use of a virtual tray by a manufacturer, distributor, medical facility, and personnel in an operating room, according to an exemplary embodiment.

FIG. 5 is a flow chart of a process for an interaction between personnel in an operating room and a virtual tray, according to an exemplary embodiment.

FIG. 6 illustrates a virtual tray user interface of the present disclosure, according to an exemplary embodiment.

FIG. 7 illustrates a virtual tray user interface in which the user has selected a plurality of items, according to an exemplary embodiment.

FIG. 8 illustrates a virtual tray user interface in which the user has used a plurality of items, according to an exemplary embodiment.

FIG. 9 illustrates a virtual tray user interface in which the user has marked a plurality of items as damaged, according to an exemplary embodiment.

FIG. 10 illustrates a virtual tray user interface in which the user has selected an option to view a bill of materials, according to an exemplary embodiment.

FIG. 11 illustrates a virtual tray user interface in which the user can select from a plurality of options for an item, according to an exemplary embodiment.

FIG. 12 illustrates a virtual tray user interface in which a unique device identifier is determined and associated with a particular item in the tray, according to an exemplary embodiment.

FIG. 13 illustrates a virtual tray user interface in which an individual item with an individual unique device identifier is selected, according to an exemplary embodiment.

FIG. 14 illustrates a virtual tray user interface in which different unique device identifiers are associated with multiples of the same type of item, according to an exemplary embodiment.

FIG. 15 illustrates a virtual tray user interface in which instructions for using the various items in the trays during a procedure are presented, according to an exemplary embodiment.

FIG. 16 illustrates a virtual tray user interface in which instructions for using the various items in the trays during a procedure are presented, according to an exemplary embodiment.

FIG. 17 illustrates a virtual tray user interface in which instructions for using the various items in the trays during a procedure are presented, according to an exemplary embodiment.

FIG. 18 illustrates a virtual tray user interface in which specific instructions for a specific step of a procedure are presented in greater detail, according to an exemplary embodiment.

FIG. 19 illustrates a virtual tray user interface in which a user can access a bill of materials for items used, according to an exemplary embodiment.

FIG. 20 illustrates a bill of materials presentable by the virtual tray user interface, according to an exemplary embodiment.

FIG. 21 illustrates a virtual tray user interface in which a user may access an administrative console, according to an exemplary embodiment.

FIG. 22 illustrates a virtual tray user interface in which a user may access individual case information, according to an exemplary embodiment.

### DETAILED DESCRIPTION

Referring generally to the figures, systems and methods for providing a user friendly interface for tracking devices and instruments are shown. The interface provides a way for tracking devices and instruments in a tray from the time the tray leaves the manufacturer to the time the devices and instruments are used. The interface allows for reliable tracking of unique device identifier information (UDI) and for simplification of every transaction involving the devices and instruments.

In the present disclosure, the interface is described with reference to bulk packaged steam sterilized devices. Such devices may be packed into a tray at the manufacturer and shipped out. In other embodiments, the systems and methods herein may be adapted to be implemented with medical implants, or devices and instruments packaged or sterilized in any other way. Further, while the present disclosure describes the implementation of the interface in a medical setting, it should be understood that the systems and methods described herein may be implementable for any type of devices in any type of setting.

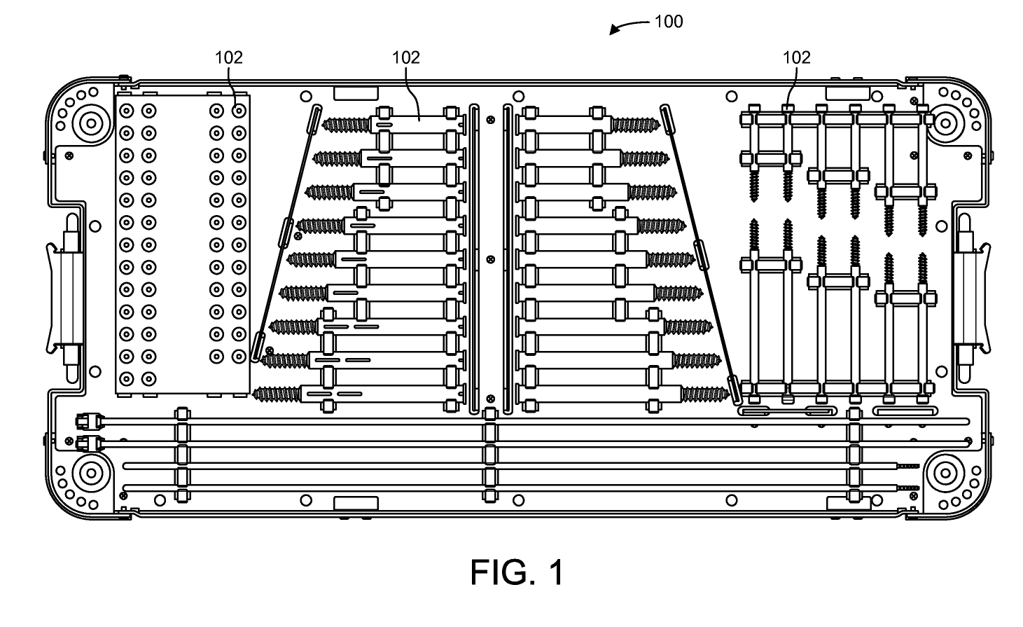

Referring to FIG. 1, an illustration of a virtual tray 100 design is shown, according to an exemplary embodiment. Virtual tray 100 is a visual illustration of an actual tray of medical items that can be used by medical personnel in a medical facility. The actual tray may generally include any type of medical tool that can be used in a procedure, any type of fastener that can be used during a procedure (e.g., bolt, nail, adhesive, etc.), or any type of implant or other object to be inserted during a procedure. It should be understood that the contents of a particular tray may vary without departing from the scope of the present disclosure. Referring generally to the disclosure, the term “item” is provided to collectively describe the various devices, instruments, tools, etc. that can be found in the tray; it should be understood that the term is not limiting. Also referring generally to the disclosure, “medical facility” may refer to any type of health care institution, a hospital, or any other establishment providing medical services.

The virtual tray 100 design shown is an example design that may be presented on a user interface to a user (e.g., a surgeon, manufacturer, distributor, etc.). In one embodiment, virtual tray 100 may look similar to the actual tray. Virtual tray 100 may be any two-dimensional or three-dimensional view illustrating the actual tray and the contents of the tray, and may be of any other design or type without departing from the scope of the present disclosure. Virtual tray 100, in various embodiments, may be shown from any angle, may be configured to rotate or provide different views, etc. As shown in FIG. 1, virtual tray 100 is a two-dimensional top-down view.

Virtual tray 100 may include visual representations for any number of items 102 in the actual tray. In the example of FIG. 1, virtual tray 100 is shown to display several different types of implants and fixation devices (in different sizes and numbers) that can be used during an operation. Virtual tray 100 is configured to be unique to the actual tray. When the actual tray is shipped from a manufacturer, virtual tray 100 may be populated with information associated with items 102 in the tray. This specific individualized data may be accessed at the manufacturer, a distributor, a sales representative, a health care institution, and in an operating room, as described below. In various embodiments, virtual tray 100 may be interacted with via a mouse, human digit, keyboard, barcode reader, RFID reader, image recognition devices, or any other type of device, on a touchscreen or any other kind of display or input device.

Unique device identifier (UDI) information may be associated with each item in virtual tray 100 and may be presented in various ways on the actual items. For example, items may be color coded, or have individual patterns or illustrations recognizable by optical character recognition (OCR), a barcode, or other methods. The UDI information may include an item or lot number specific to the item.

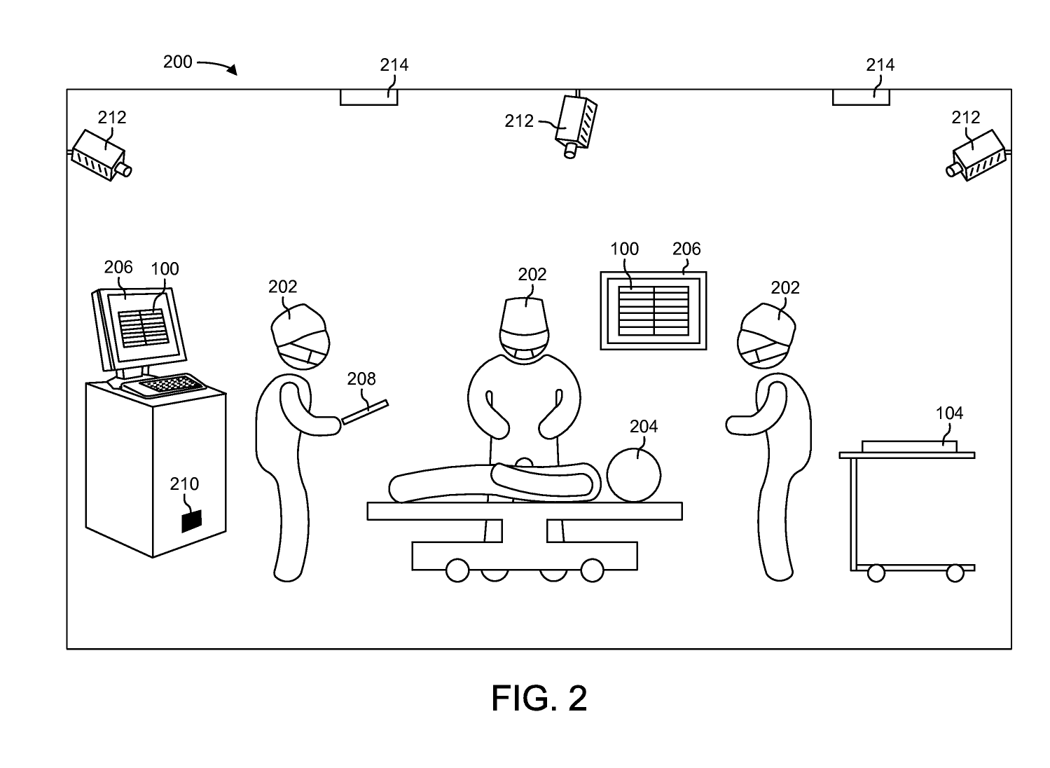

Referring to FIG. 2, an example operating room 200 is shown. Operating room 200 is shown as an example environment in which the virtual tray system of the present disclosure can be implemented. In the example of FIG. 2, operating room 200 is an environment in which a surgical procedure is performed in which one or more items from a tray will be used. The virtual tray system is used to track the usage of the one or more items. While the systems and methods to be described herein are described with respect to a procedure in an operating room, it should be understood that in other embodiments, the systems and methods described herein may be implemented in any other location.

In the embodiment of FIG. 2, operating room 200 is shown occupied by one or more personnel 202 (e.g., a surgical team, surgeons, nurses, or other personnel) and a patient 204. Operating room 200 may include various equipment for assisting personnel 202 with a medical procedure to be performed on patient 204. For example, operating room 200 includes displays 206, which may be used to display patient information, provide video, or otherwise. As another example, operating room 200 includes one or more remote devices 208, belonging to an individual or the room in general. Remote devices 208 may be, for example, cell phones, tablets, laptop computers, or other handheld devices, and may display information in a similar manner to displays 206. In some embodiments, one or both of displays 206 and remote devices 208 are touchscreen capable, and may be displays of any type of configuration (e.g., LCD, CRT display, etc.). In some embodiments, displays 206 may be in a fixed location or may be temporarily or permanently positioned in operating room 200 by personnel 202. While not shown in FIG. 2, operating room 200 may further include other devices for providing sensory feedback to personnel 202.

Virtual tray 100 may be displayed on one or more displays 206 and/or devices 208. This allows virtual tray 100 to be available to one or more personnel 202 in operating room 200 during an operation. Each individual may have a unique ID associated with him or her, allowing specific interactions with virtual tray 100 as described below.

In some embodiments, when personnel interacts with an actual tray 104 of items in operating room 200 (or elsewhere between the manufacturer and the operating room in other embodiments), an interaction involving virtual tray 100 and displays 206/device 208 may be initiated. For example, when a user touches or removes an item in tray 104, or selects an item via display 206/device 208, specific information relating to the item may be accessed on the virtual tray (i.e., displayed on the display/device of the surgeon). As another example, touching or removing an item from tray 104 may cause the item to be added to a bill of materials or invoice that charges the medical facility for use of the item. As another example, upon use of an item in tray 104, the tray inventory may be updated. Tray 104 may generally include a monitoring system and/or one or more monitoring devices for detecting the use of an item, may include an interface for allowing personnel 202 to identify an item being used, or may detect the usage of an item in any other way. The management of the various interactions with tray 104 is described in greater detail below.

Operating room 200 may include a monitoring system 210 which may be implemented either in a computing device located in operating room 200 or remotely from operating room 200. Monitoring system 210 may monitor the activity in operating room 200. For example, monitoring system 210 may monitor the use of one or more medical devices, such as items from tray 104, during an operation. Monitoring system 210 may be used in conjunction with or instead of a monitoring system of tray 104 for detecting the use of one or more items in the tray. Monitoring system 210 may be connected to a plurality of devices, such as cameras 212, RFID readers 214, or other devices, configured to detect the usage of items in tray 104. For example, one or more items may have a RFID tag attached to it that is detectable by RFID reader 214. Monitoring system 210, cameras 212, RFID reader 214, and other monitoring devices and methods may further be used in operating room 200 for other functionality not related to the use of items in tray 104.

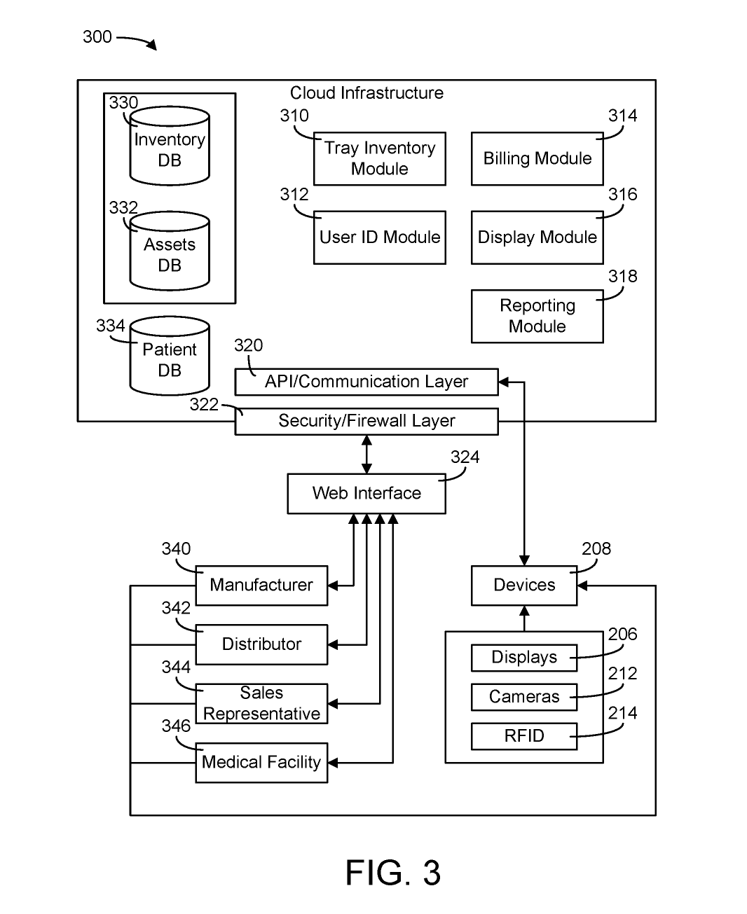

Referring now to FIG. 3, a block diagram of a virtual tray system 300 is shown, according to an exemplary embodiment. Virtual tray system 300 is shown to generally include various modules, databases, devices, and other systems that may communicate with one another via a cloud-based architecture. The various modules and databases shown in virtual tray system 300 may maintain virtual trays for a plurality of users who wish to access the virtual trays, generate an invoice for used items, provide instructions relating to the use of items in a tray, etc. The databases of virtual tray 300 may store inventory information for a virtual tray, asset information, patient information for a patient for which a particular tray will be used in an operation for the patient, etc. Virtual tray system 300 may be accessed by users such as a manufacturer 340, a distributor 342, a sales representative 344, or users at a medical facility 346. Virtual tray system 300 may further receive input from devices 208 (e.g., user devices) and operating room devices (such as displays 206, cameras 212, RFID readers 214, etc.).

Referring generally to the disclosure, activities described as being executed by virtual tray system 300 may generally include activities executed by the individual modules and databases as described below. The term “virtual tray system” is used to describe the collective activities of the modules and databases, and it should be understood that such activities may be executed by any number of individual modules and databases.

In some embodiments, virtual tray system 300, and more particularly the modules and databases shown, may be implemented at a single server, at one or more servers, or at any number of other devices within “the cloud”. Virtual tray system 300 is shown to generally include an API/communication layer 320 configured to facilitate communications between the modules and databases and the various users and devices attempting to access a virtual tray. Virtual tray system 300 is further shown to include a security/firewall layer 322 configured to secure the communications between the modules and databases and the users and devices. Layers 320, 322 may be configured to receive communications directly from the users and devices, or the users may communicate with the modules and databases via a web interface 324 that allows a user to enter information about a tray.

It should be understood that the architecture described herein is provided by way of example only, and that virtual tray system 300 may be implemented in various ways. In the cloud-based embodiment of FIG. 3, various modules are shown which may have various interdependencies and interconnections with other modules and databases. Multiple databases are shown that allow sensitive content (e.g., patient information) to be more secure (e.g., being stored remotely from the various modules). In another embodiment of virtual tray system 300, a central server may be configured to receive input from the variety of users and devices, and may include all the modules and databases shown.

Each module of virtual tray system 300 may be implemented by any number of servers, virtual machines, devices, etc. Different modules may be implemented at the same server or machine or different servers or machines. Each server or machine may generally include a processing circuit including a processor and memory to execute the activities of the module. The processor may be implemented as a general purpose processor, an application specific integrated circuit (ASIC), one or more field programmable gate arrays (FPGAs), a group of processing components, or other suitable electronic processing components. The memory is one or more devices (e.g., RAM, ROM, flash memory, hard disk storage, etc.) for storing data and/or computer code for completing and/or facilitating the various user or client processes, layers, and modules described in the present disclosure. The memory may be or include volatile memory or non-volatile memory. The memory may include database components, object code components, script components, or any other type of information structure for supporting the various activities and information structures of the present disclosure. The memory is communicably connected to the processor and includes computer code or instruction modules for executing one or more processes described herein.

An inventory module 310 is configured to track the inventory of an actual tray, for the virtual tray generated by virtual tray system 300. An individual tray may include a unique identifier for the tray along with a unique identifier of each item in the tray. Inventory module 310 may be configured to maintain an inventory of each tray (using the tray unique identifier), and more particularly a quantity of each item available in the tray. Inventory module 310 may receive an input from any of manufacturer 340, distributor 342, sales representative 344, or a user in medical facility 346 regarding a change in status of the tray (e.g., the addition or removal of an item), and may update the inventory of the virtual tray in real-time or near real-time. In various embodiments, virtual tray system 300 and inventory module 310 may track any number of trays from any number of manufacturers. In some embodiments, inventory module 310 may maintain an inventory database 330 for storing tray inventory information for the plurality of trays.

In some embodiments, tray inventory module 310 updates an inventory of a tray based on an input from a manufacturer 340. At manufacturer 340, a tray may be prepared and sent out to a distributor. The tray may be prepared based on an order from a medical facility or personnel, prepared for a specific operation with a patient or a specific type of operation, or may be a tray generally prepared by manufacturer 340 for general use. Manufacturer 340 may place a plurality of items in the tray, such as one or more implants to be inserted into a patient during an operation, one or more tools for use during the operation, and other devices. Each item placed in the tray may be recorded by manufacturer 340 to allow the item to be identified by tray inventory module 310. For example, each item may be associated with a unique device identifier that can be detected by another user or device. The unique device identifier may be a numerical value, a name, a visual indicia (e.g., color coding or an image), etc. Therefore, when the item is used, it can be decremented from the inventory of the tray. Further, each item may be assigned a particular location or storage method within the tray.

In some embodiments, manufacturer 340 may attach specific content to each item. For example, the content may be instructions for using the item, warnings or contraindications associated with the item, an expiration date of the item, a date of manufacture of the item, a fatigue life of the item, or any other useful information. The information is associated with each item via the item identifier. Further, each item is associated with the tray in which it is housed.

Tray inventory module 310 may receive input from manufacturer 340 regarding the contents of a tray, as the manufacturer is setting up the tray or after. In some embodiments, manufacturer 340 may scan identifiers for each individual item to be placed in the tray, and the device scanning the items may relay the information to tray inventory module 310. In some embodiments, the tray may include one or more sensors or devices for tracking the items placed within the tray, and such information may be provided via the sensors or devices to tray inventory module 310.

In some embodiments, tray inventory module 310 updates an inventory of a tray based on an input from a distributor 342. Distributor 342 may receive a tray from manufacturer 340, and be generally responsible for distributing (e.g., selling) trays to a plurality of medical facilities 346, restocking trays, and the like. At distributor 342, for example, implants in the tray may be repopulated with devices shipped from manufacturer 340. In some embodiments, the unique information associated with the items may already be associated with a specific tray location by manufacturer 340 and may be confirmed by distributor 342. In other embodiments, the unique information associated with the items may be assigned a specific tray location at distributor 342. In such embodiments, distributor 342 may provide tray location data for each item to tray inventory module 310 in order to keep the virtual tray up to date.

In some embodiments, tray inventory module 310 updates an inventory of a tray based on an input from a user at medical facility 346 (or other central receiving or supply department that receives a tray for use in an operating room at the medical facility). At medical facility 346, the actual tray may be received and compared to the virtual tray (which is accessible through an online user interface as described below) in order to confirm the inventory in the tray and record deficiencies (i.e., identify broken or missing items). Personnel at medical facility 346 may further verify product usage after an operation, as described below.

In some embodiments, at medical facility 346, it is a large facility with many consigned products. Therefore, the tray at medical facility 346 may be accessed by users such as sales representatives 344 (selling items and other devices to the institution) and facility employees. In some embodiments, tray inventory module 310 may be configured to receive an input when a sales representative 344 repopulates the tray in concert with the manufacturer and distributor. For example, the medical facility may order additional items through sales representative 344 from a manufacturer and distributor to be placed in the tray. Tray inventory module 310 receives an indication of the transaction and updates the inventory of the tray in response.

In some embodiments, tray inventory module 310 updates an inventory of a tray based on an input from a device 208 in an operating room 200. Virtual tray system 300 provides a virtual tray for display on a screen in operating room 200 (as described by display module 316 below). The virtual tray may be accessible to any personnel in the operating room (e.g., a scrub tech, circulating nurse, sales representative, etc.) via a device 208. The personnel can use device 208 to access information related to the tray and the items in the tray. Tray inventory module 310 may receive input from the devices in operating room 200 relating to an interaction with the tray. This input may be from personnel or detected by a camera 212 or sensor 214. For example, a sensor on the tray may detect the removal of an implant from the tray and relay the information (through device 208) to tray inventory module 310, which may then update the inventory of the tray accordingly.

In one embodiment, the input received by tray inventory module 310 may be from a RFID reader 214. RFID reader 214 may generally be configured to detect the use of an item in the tray by reading a RFID tag that is coupled to the item. RFID reader 214 may detect when the item is being moved, and to associate the movement with usage of the item. Tray inventory module 310 may receive the detection by RFID reader 214 and update the virtual tray accordingly.

Each person accessing the virtual tray via virtual tray system 300 may have a unique ID associated with him or her. A user ID module 312 may be configured to manage user interactions with the virtual tray system based on the user ID. Each person may generally have a unique ID (or other more general identifier) that identifies the user to user ID module 312. User ID module 312 may allow different levels of interactions, or specific interactions, with the virtual tray depending on the user ID. For example, some users (e.g., surgeons) may be able to access information relating to the use of an implant or tool during a surgical procedure. The surgeon may be able to touch a touchscreen displaying the virtual tool in order to view specific information or diagrams. As another example, some users (e.g., sales representatives) may be able to access information relating to the available inventory or stock of an item that the user wants to sell to the hospital, via the virtual tray. As yet another example, a surgeon may be able to access patient data via patient database 334 during an operation or before the operation, preparing for the operation.

In some embodiments, user ID module 312 may track access of the virtual tray by the various users. For example, user ID module 312 may be configured to store a history of who interacted with the virtual tray, who removed or added items to the virtual tray, and so forth. This information may be stored in inventory database 330 (allowing tray history data to be stored with other tray information), assets database 332, or may be stored in another local or remote database or data store.

In some embodiments, a user may touch or remove an item from the tray in operating room 200. The interaction with the item may be detected by a camera, sensor, or the tray, and sent to tray inventory module 310. In other embodiments, a user can indicate use of an item through the user interface (described below). In response, a bill of materials may be generated for the use of the item in a surgical procedure. For example, if an implant was removed, an invoice charging the medical facility for the use of the implant in a procedure may be generated. A billing module 314 is shown for facilitating such a transaction relating to the use of the item. In some embodiments, billing module 314 may automatically generate a purchase order and sales invoice in response to the use of an item with a particular UDI. Billing module 314 may generate the bill of materials in conjunction with user ID module 312 (e.g., user ID module 312 may identify the personnel using the item in question and may determine whom to charge for use of the item and which manufacturer to provide the revenue). Billing module 314 may be configured to generate an invoice in real time (e.g., creating the invoice as tray inventory module 310 receives indication of the use of the item, and as the virtual tray is updated), or may generate an invoice on a schedule or when requested (e.g., by a user, the medical facility, etc.).

A transaction initiated by billing module 314 may require more than one person or entity to confirm. For example, the use of the item in a procedure may be confirmed by personnel in operating room 200 after detection of the use of the item. Billing module 314 may work in conjunction with the other modules of virtual tray system 300 to verify the use of the item and whom to charge for the usage.

A display module 316 may generally be configured to generate the visual tray for display on a device. Display module 316 may generate an image (e.g., an avatar) used to represent each item of a tray. For example, referring again to FIG. 1, display module 316 may generate the virtual tray display, creating a visual representation for each item that generally looks like the actual item. Display module 316 may further use images, icons, text, and other content representative of the contents of the tray. In some embodiments, the virtual tray may be made to look as similar to the actual tray as possible (e.g., as shown in FIG. 1). In other embodiments, a more generic version of a visual tray may be generated by display module 316 for a manufacturer (i.e., providing a generic design that manufacturers can fit their devices into as an expense saving action). It should be understood that the visual features provided by display module 316 may vary according to various exemplary embodiments.

Virtual tray system 300 is shown to include a reporting module 318. During an operation at medical facility 346, one or more items of a tray may be used. Reporting module 318 may be configured to generate a report detailing the usage of the one or more items, and to associate the items with a particular patient, case, or operation as a result. This may allow virtual tray system 300 to generally track and provide information relating to the usage of a particular item. Such information may be stored in a database 330, 332, or 334.

Virtual tray system 300 is shown to include an inventory database 330, assets database 332, and patient database 334. In various embodiments, virtual tray system 300 may include any number of databases storing information relevant to the virtual trays. Inventory database 330 may generally store inventory information for each tray. Assets database 332 may generally store information regarding assets available to the personnel in the trays at the medical facility. Patient database 334 may generally store information for each patient, such as a procedure to be performed on the patient and the trays and items necessary for the procedure. For example, using patient database 334, a surgeon (or other personnel) may access all information needed for a procedure, including the one or more trays and items therein that are needed for the procedure.

In some embodiments, virtual tray system 300 may facilitate communications between personnel, distributors, and manufacturers based on the tray. For example, comments relating to the tray or the items therein may be provided by surgical personnel to be provided to the manufacturer, distributor, a central supply station, or a regulatory agency. The comments may generally relate to problems or questions related to the items in the tray. This may allow the surgical personnel to access and/or provide information that results in a corrective or preventative action.

In some embodiments, virtual tray system 300 may portray multiple virtual trays from different manufacturers on one or more screens, or may display virtual trays specific to a facility. The various virtual trays may be used to gather information from surgical personnel, the health care institution, or another end user that may be used by the manufacturer to set up the tray. The various virtual trays may further be presented when the medical facility or other end user is ordering a tray. Further, virtual tray system 300 may facilitate communications between the medical facility and manufacturer (or other users) during a transaction, allowing the manufacturer to customize the tray (i.e., provide appropriate items in the tray, provide items for a specific procedure or patient, etc.) for the facility. The display of one or more virtual trays by virtual tray system 300 may facilitate such a process, allowing the end user to access any information from the manufacturer regarding the tray or from virtual tray system 300 (e.g., information stored in inventory database 330).

In some embodiments, virtual tray system 300 may allow a virtual tray to be accessible to an online specialist. For example, during a surgical procedure, a surgeon or other personnel may select an item and ask for assistance related to the item. The personal may then be put in contact with an online specialist remotely located from the operating room, who is identified as being able to assist based on the item. The virtual tray may be accessible to the online specialist, allowing the specialist to assist in the surgical procedure.

In some embodiments, virtual tray system 300 may provide a training virtual tray for training for personnel at a medical facility. The training tray may be used to familiarize personnel with the devices, implants, and other items in the virtual tray, and to provide information relating to a procedure associated with the devices and implants. Virtual tray system 300 may include a database or other data store (or be connected to the database or other data store) storing such information, or may retrieve the information from inventory database 330.

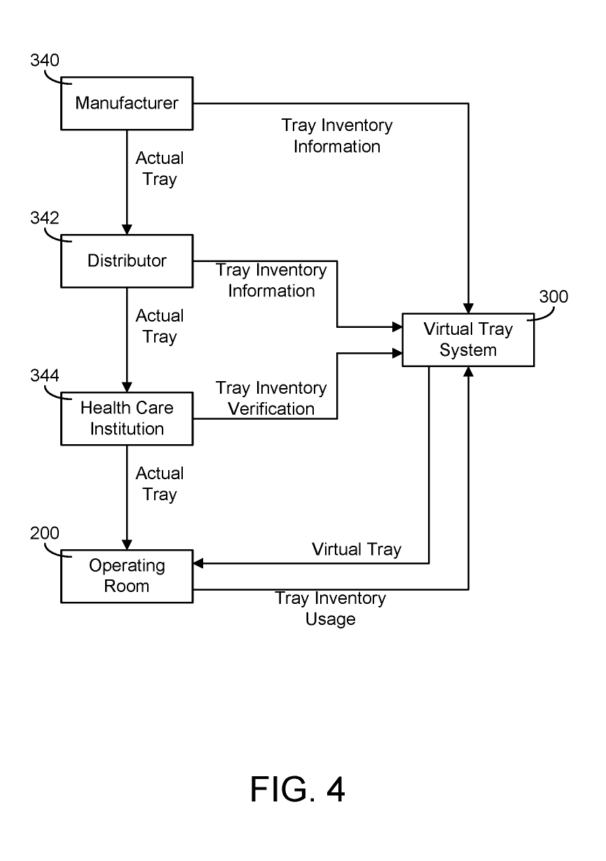

Referring now to FIG. 4, the use of a virtual tray by a manufacturer 340, distributor 342, medical facility 346, and personnel in operating room 200 is shown in greater detail. More particularly, FIG. 4 details the various types of interactions that may occur between users and the modules of virtual tray system 300. Virtual tray system 300 may create a virtual tray for an actual tray as manufacturer 340 is setting up the tray. Setting up the virtual tray may include receiving a tray identifier and a UDI for each device placed in the tray by manufacturer 340. After the tray is set up by manufacturer 340, the tray is provided to distributor 342. Distributor 342 receives the tray and repopulates the tray, and may work in conjunction with virtual tray system 300 to properly place the items in the actual tray.

The actual tray is provided by distributor 342 to a medical facility 346 (e.g., a hospital). Upon receiving the tray, medical facility 346 may compare the actual tray to the virtual tray (provided by virtual tray system 300) to confirm the inventory. The actual tray may be moved to an operating room 200 in which the items of the tray will be used. Upon detection of usage of an item in the tray, an indication may be sent to virtual tray system 300, and the virtual tray may be updated accordingly. Further, virtual tray system 300 may provide notification (via changing the virtual tray) of the change to manufacturer 340, distributor 342, and medical facility 346.

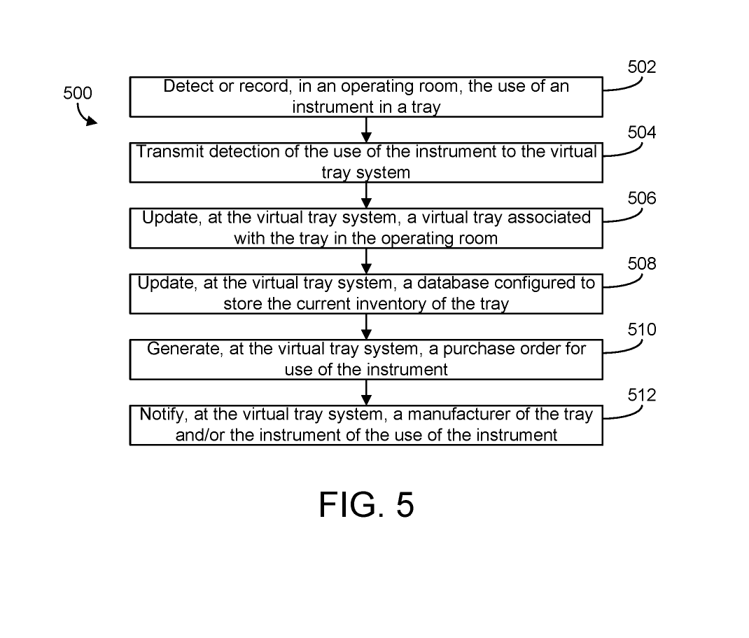

FIG. 5 is a flow chart of a process 500 for an interaction between personnel in an operating room and a virtual tray, according to one embodiment. Process 500 includes detecting or recording the use of an item in a tray in an operating room (block 502). For example, block 502 may include detecting the removal of an item in the tray. As another example, block 502 may include detecting that a user has touched the item. As yet another example, a sensor or other device in the operating room may detect the usage of the item (e.g., by scanning a tag of the item). As yet another example, a user in the operating room may select the item via a virtual tray display provided on a touchscreen device in the operating room. Process 500 includes transmitting the detection of the use of the item to a virtual tray system (block 504). While the embodiment shown herein relates to detecting and transmitting the use of an item, the same process may also apply to detecting and transmitting information regarding damage to an item.

Process 500 further includes updating a virtual tray associated with the tray in the operating room (block 506). For example, block 506 may include removing an icon associated with the item in the virtual tray display. Process 500 further includes updating an tray database configured to store the current inventory of the tray (block 508).

In some embodiments, process 500 includes generating a purchase order for use of the item (block 510). In other words, the personnel or the health care institution in which the operating room is located may be charged for use of the item. Process 500 further includes notifying a manufacturer of the tray and/or the item of the use of the item (block 512). This may cause the manufacturer to receive payment for use of the item, alert the manufacturer or other user to provide a replacement item, or otherwise.

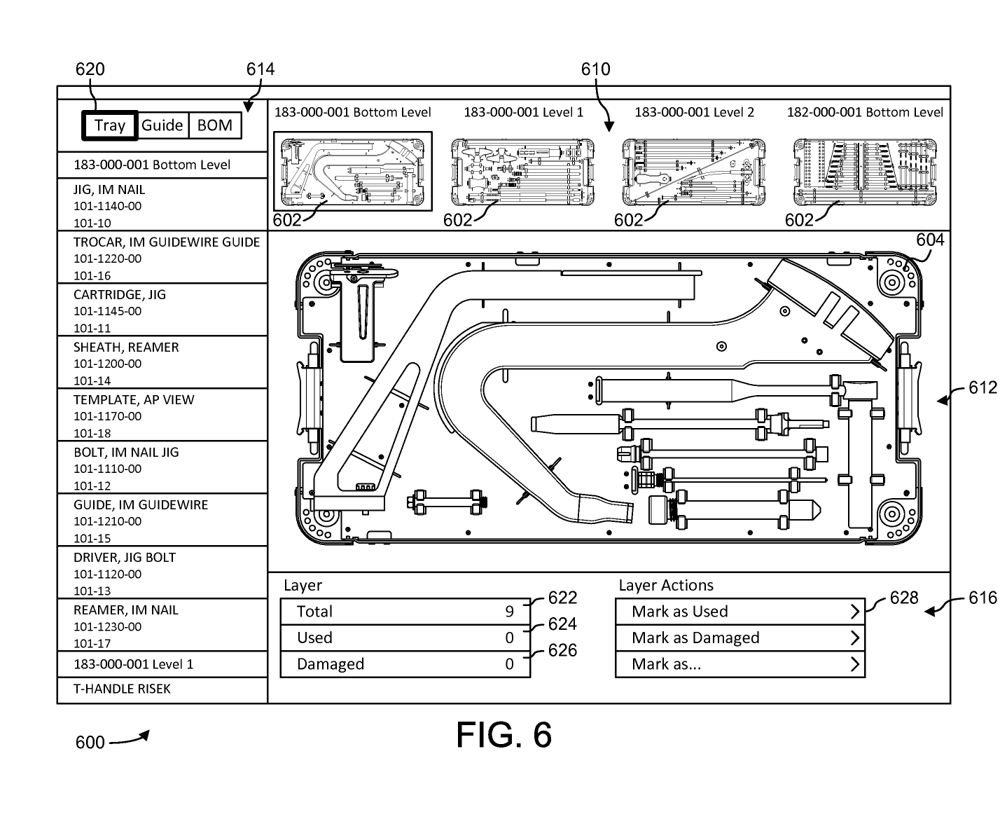

Referring now generally to FIGS. 6-22, various example user interfaces are shown. The user interfaces illustrate example virtual trays that may be generated by the virtual tray system of the present disclosure. In FIGS. 6-22, the example user interfaces are shown provided on a mobile device such as a mobile phone or a tablet; it should be understood that this example is not limiting, and that the virtual tray may be presented in various ways on various displays without departing from the scope of the present disclosure. In the example of FIGS. 6-22, the user interface may be presented on a touchscreen allowing a user to directly select an option; in other embodiments the systems and methods described below may be adapted to allow for any other type of user input (e.g., mouse, keyboard, voice input, etc.).

Referring to FIG. 6, an example user interface 600 is shown. An actual tray assembly may include multiple trays or “levels”, i.e., each level being its own tray with different items. In the examples of FIGS. 6-22, four different trays are shown, each tray part of a tray assembly. The user may be able to select different trays from the tray assembly at any time via user interface 600. For example, in FIGS. 6-22, the user may select any particular tray 602 (see, for example, the four trays 602 shown across the top of the user interface 600) to cause a larger display of the tray to appear in user interface 600. This selection may generally indicate which tray is in use by the user. In various other embodiments, the user interface may display trays from multiple tray assemblies, only trays with items that a user may need during the operation, or otherwise.

User interface 600 may generally include a first portion 610 or section in which the multiple trays available to a user are shown. The user may select a tray to cause a second portion 612 of the display to show the selected tray 604. User interface 600 also includes a third portion 614 in which, in the example of FIG. 6, a list of items in tray 604 is shown. The “tray” option 620 is shown selected in FIG. 6, which causes a list of items in the tray to be presented (the “guide” and “BOM” options are described below). Tray 604 is shown to include an identifier (“183-000-001”) and each item is shown to include an UDI (e.g., “101-1140-00”). User interface 600 also includes a portion 616 in which general information about and options for tray 604 are provided. For example, the number of items in the tray (field 622), the number of items used so far (field 624) and the number of items damaged (field 626) are displayed. Further, the user may be able to select an option 628 to mark an item as used, damaged, or otherwise. The layout shown in FIG. 6 (and subsequent figures) may vary according to various embodiments, and the example layout of the tray information as shown is not limiting.

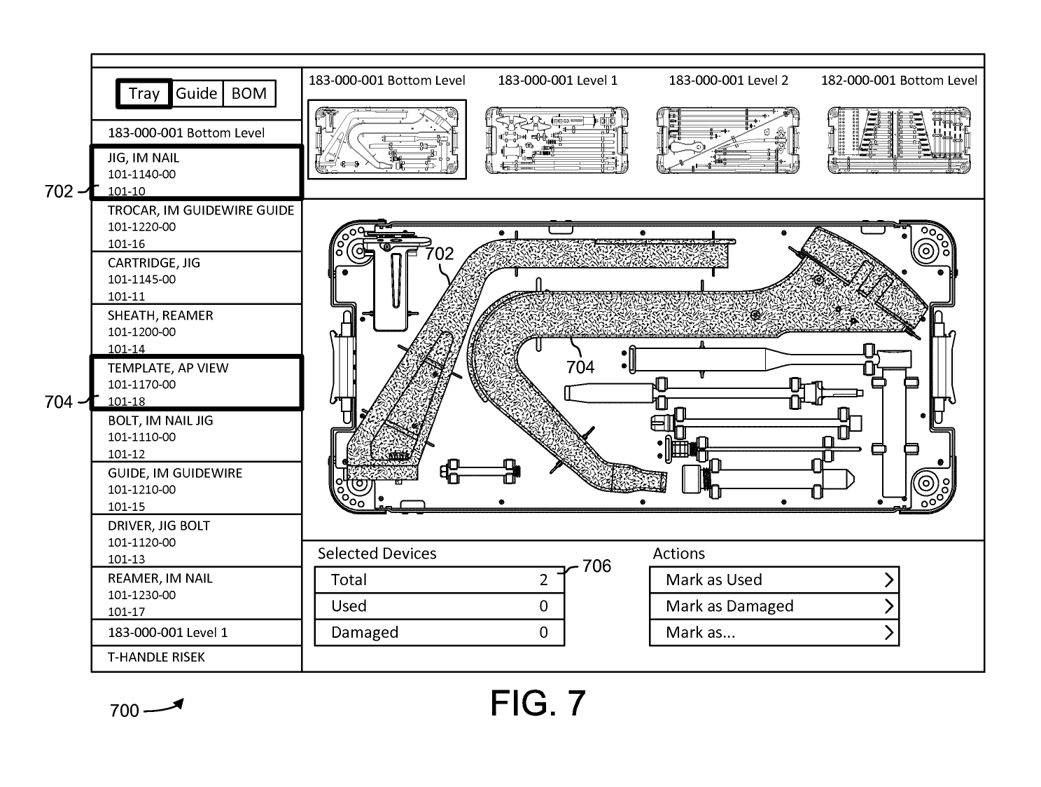

Referring to user interface 700 of FIG. 7, the user has selected two of the items 702, 704. Both items 702, 704 are shown highlighted in the virtual tray. In various embodiments, the items, when selected, may be highlighted in any way (e.g., shaded, brightened, shown in any color or combination of colors, may be outlined in any way, may be flashing, etc.). Further, the items are shown highlighted within the list of items. This highlighting, again, may be done in any way to differentiate the selected items from the other items. In various embodiments, an item may be selected either by selecting the virtual item shown in portion 612 or by selecting the item from the list in section portion 614. The item may then be highlighted in both portions. Further, a selected device field 706 may be displayed indicating, in this example, that two items have been selected by the user.

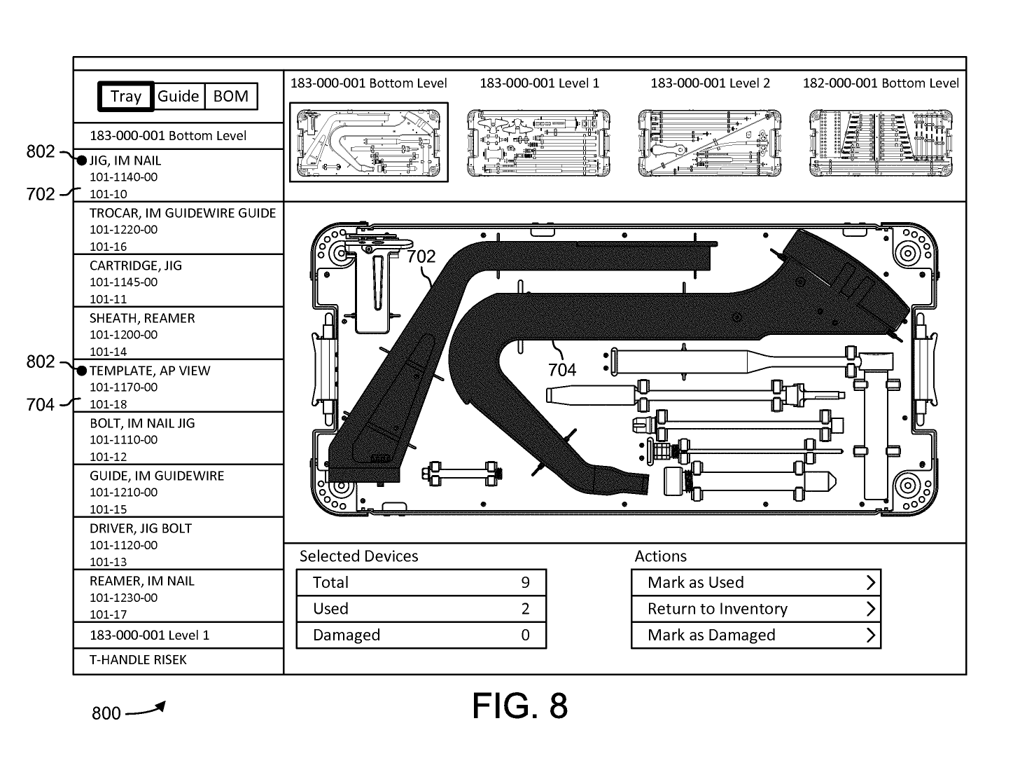

Referring to user interface 800 of FIG. 8, the user has now marked the two selected items 702, 704 as used (e.g., via selecting option 628). A visual indicia 802 is shown placed next to both items in the list, indicating that the items have been used in a procedure. In various embodiments, the use of an item may be marked in any other way (e.g., an icon of any size, shape or color placed next to the item on the list, the highlighting, shading, or other manipulation of the field in which the item is listed, etc.). Further, the two items 702, 704 may be highlighted in the virtual tray, in a different shading, color, etc., that indicates that the items are no longer available.

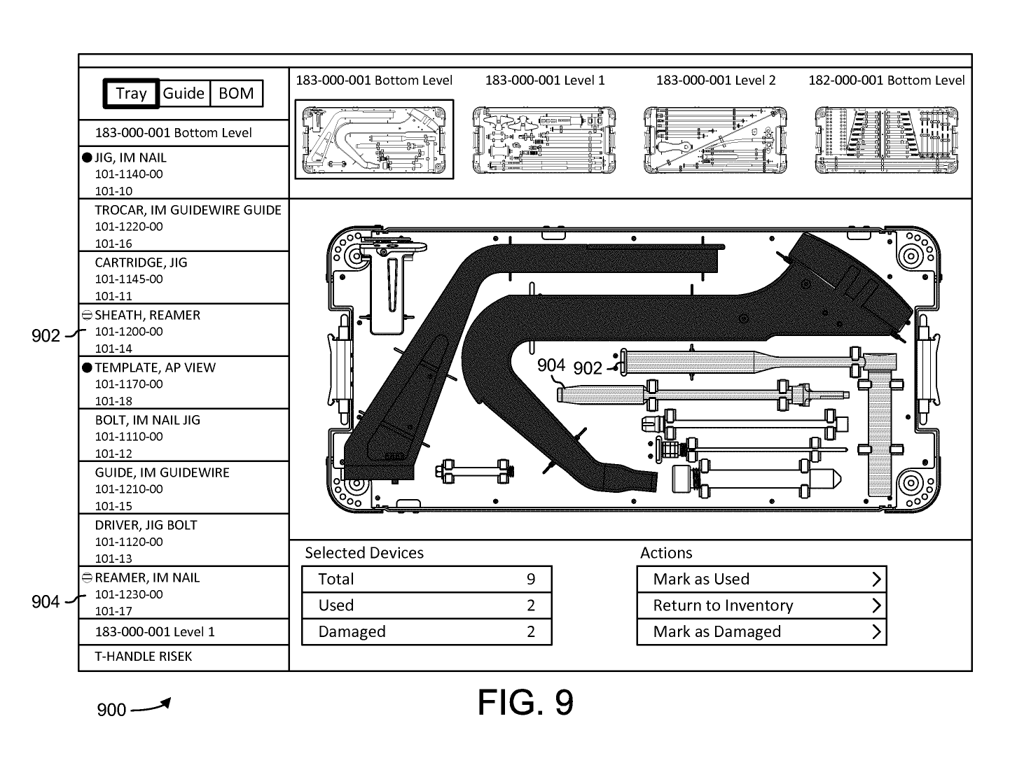

Referring now to user interface 900 of FIG. 9, the user may also mark an item as damaged. As shown in FIG. 9, two items 902, 904 are marked as damaged by the user. Items 902, 904 are shown highlighted in the virtual tray (but highlighted differently from items 702, 704) and in the list of items using a different visual indicia.

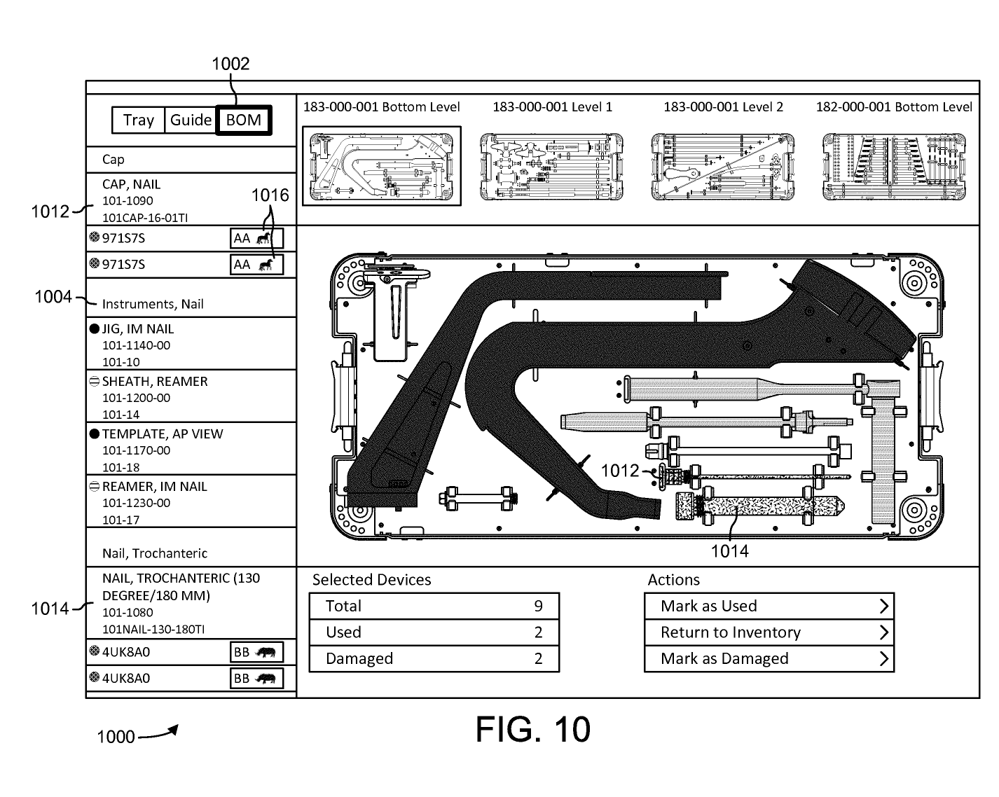

Referring to user interface 1000 of FIG. 10, the user has selected the bill of materials (BOM) tab 1002 instead of the tray tab. In the list, the used and damaged items 1004 are shown listed together. In the example of FIG. 10, the items are nail instruments that can be used to place and secure a nail in a patient. Since these items have been used (and/or damaged), the medical facility may be billed for use of the items. By selecting BOM tab 1002, the user may view the list of items for which they will be billed.

As shown in user interface 1000, two additional items 1012, 1014 are selected and highlighted in list 1004 and in the virtual tray. In the example of FIG. 10, the items are a nail cap and a nail that can be used by the items already selected. Each item is shown to have a UDI, but have multiple items listed under it (i.e., there is more than one of the item in the tray). Each individual item is associated with a visual indicator 1016, which will be described in greater detail below. An item may also be selected by imprinted characters, such as a lot number, which can be selected from the tray inventory list.

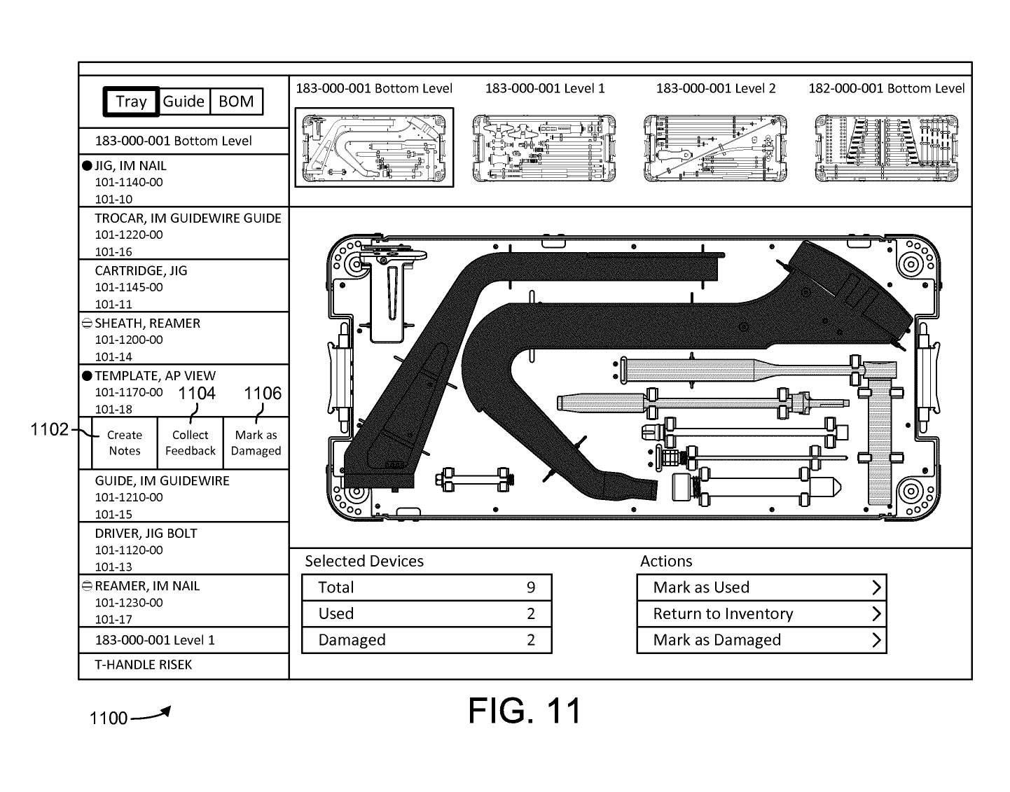

Referring now to user interface 1100 of FIG. 11, an additional way of allowing a user to provide an input to the virtual tray system is shown. If the user selects an item in the list of items, multiple options may be presented to the user. The selection of the item may simply be a touch on the touchscreen of the item in the list, a swipe left or right on the touchscreen, or otherwise. The multiple options presented include an option 1102 for creating notes (e.g., notes about the item that can be stored in a database at the virtual tray system), an option 1104 for collecting feedback (e.g., providing and receiving information to/from other users relating to the item), and an option 1106 to mark the item as damaged. Other options may also be provided to a user in this manner. The user may be able to adjust his or her personal settings to choose the options available in this way.

The user interfaces of FIGS. 6-11 generally illustrate various ways for a user to interact with the virtual tray during, for example, an operating procedure. For example, the embodiments of FIGS. 6-11 illustrate how a user may select items for use, how to mark items as being used or damaged, and the like. The virtual tray system may allow for any method of highlighting selected items, marking items as used or damaged, or for providing any other visual indicia of the current status of the tray.

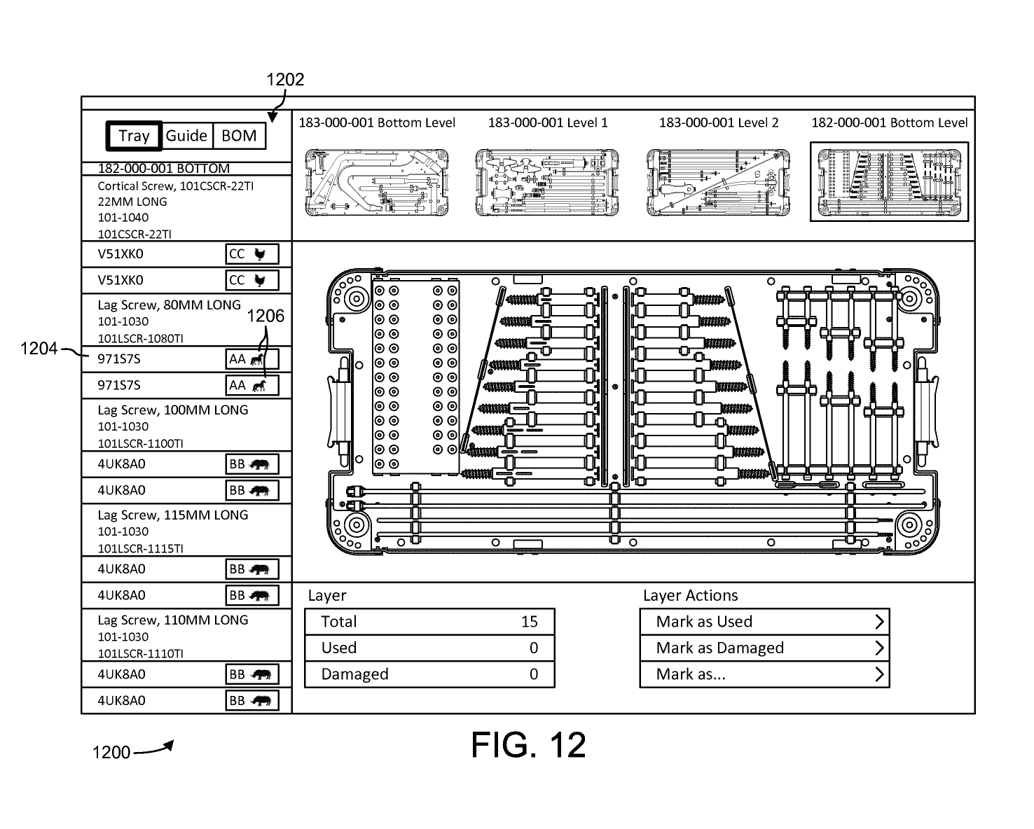

Referring generally to FIGS. 12-14, UDI tracking features of the virtual tray are described in greater detail. As described above, each item in the actual tray may include a UDI (or other identifier) that marks the item as unique from other items. In various embodiments, the items may include a tag readable by a RFID reader or other sensor, may be color coded, may portray a recognizable object, such as the animals shown, or may be marked in any other way. When an individual item is used by a user, the UDI of the item may be determined and used by the virtual tray system to mark the item as used or damaged, to provide options to the user relating to the item, and the like.

Referring to user interface 1200 of FIG. 12, a virtual tray with various screws is shown. A list 1202 of screws available in the tray is shown. In the example of FIG. 12, there may be multiple screws with the same dimensions (e.g., multiple screws that are 80 mm long, 100 mm long, etc.). Each type of screw may have its own identifier 1204 (e.g., “971S7S”, “4UK8A0”, etc.). Further, each individual screw may have a visual indicator 1206. For example, for the 80 mm lag screw shown in FIG. 12, a visual indicator labeled “AA” and marked with a horse is shown for each screw. Visual indicator 1206 may further or alternatively include any type of text, number, icon, or picture, and may include any type of color or shading. This identifier may be adjusted as one or more of the screws are used or associated with a particular step or procedure, as described below.

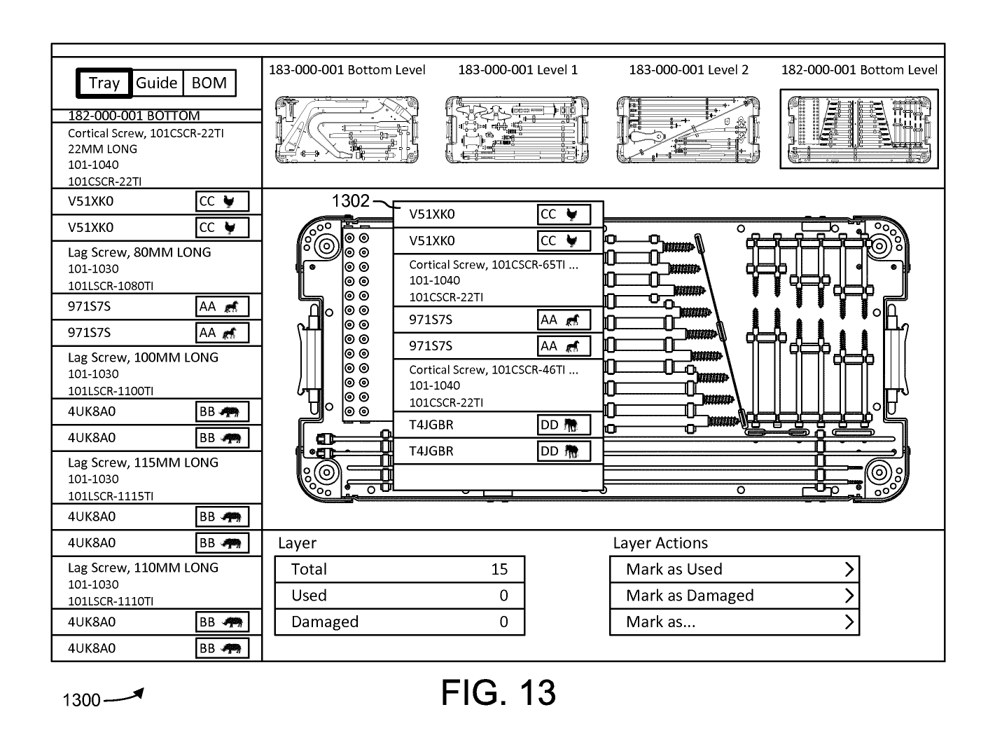

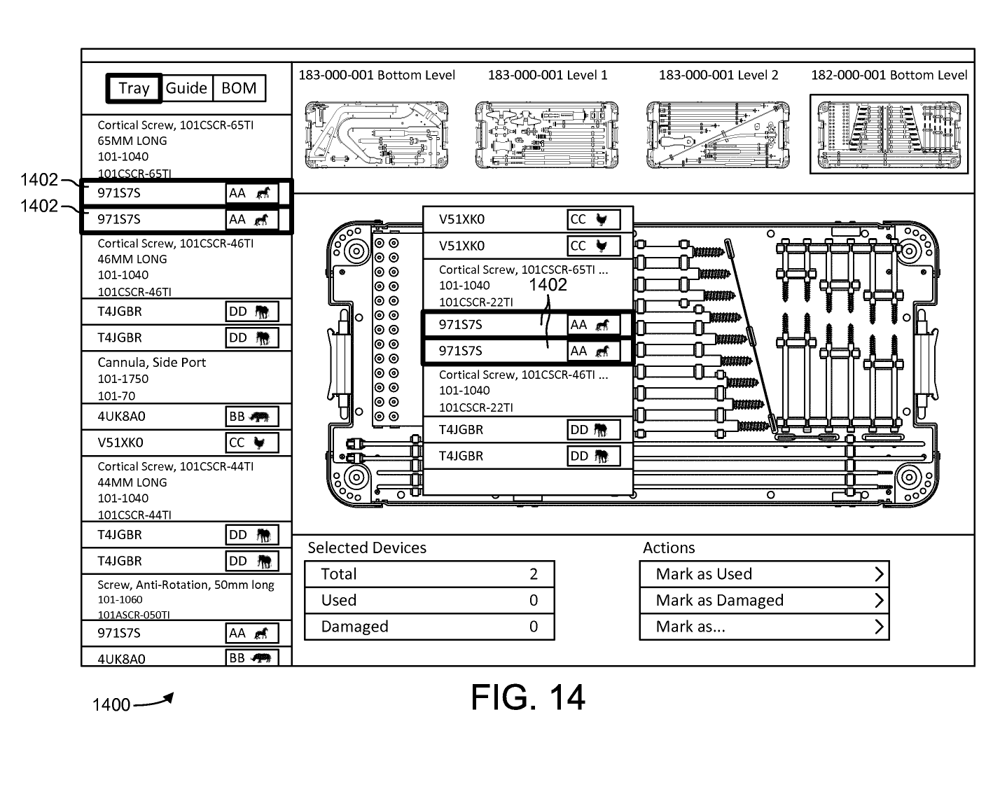

Referring to user interface 1300 of FIG. 13, an additional list 1302 may be displayed for the user. In some trays, a screw caddy or other similar device may hold multiple instruments or implants in the tray. Because of interface constraints, all of the items contained in the caddy may not appear on the item list, so the user may click anywhere on that element and be presented with a separate list of available items contained in the caddy. This list will contain information about the various devices, including device names, identifiers, visual indicators, and any other information relevant to the item. Referring to user interface 1400 of FIG. 14, the user has selected items 1402 for use, and those items are highlighted by the virtual tray system.

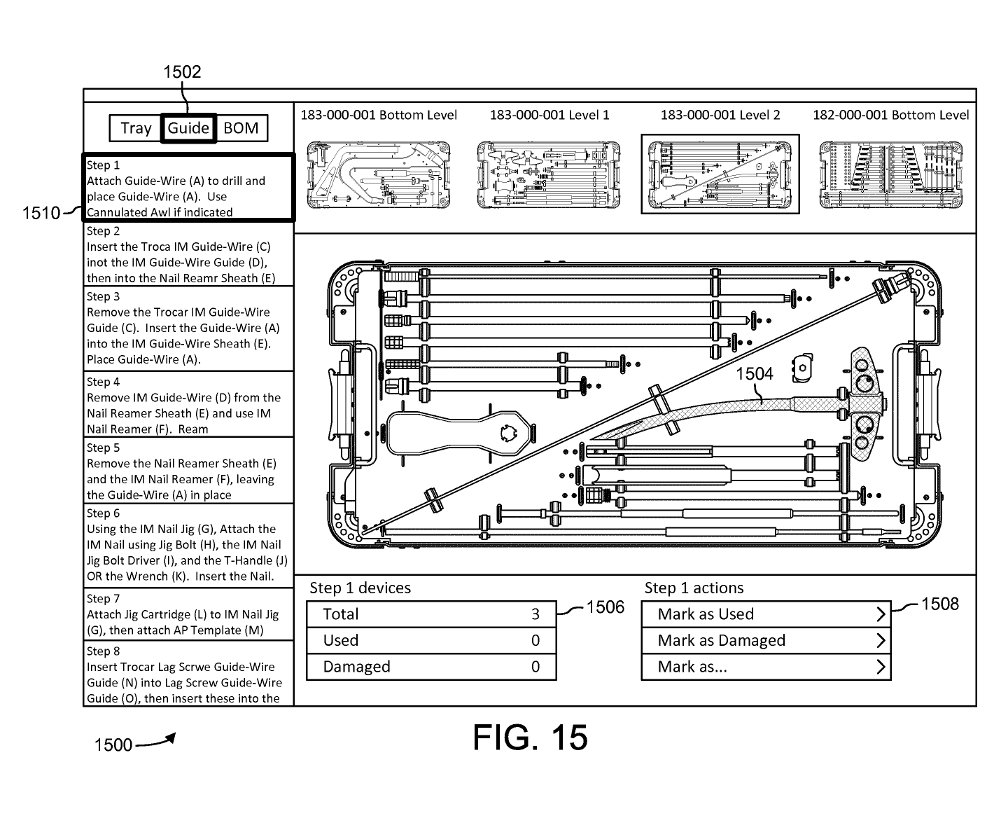

Referring generally to FIGS. 15-18, device education features of the virtual tray are described in greater detail. As described above, a user using the virtual tray may request and receive information relating to the various items in the virtual tray and actual tray. For example, the user may select an item and may be presented with information about how to use the item in a procedure. As another example, the user may request information related to a specific procedure, and the virtual tray system may identify the steps of the procedure as well as each individual item to be used in the procedure. In various embodiments, the virtual tray provides a guide, such as a surgical technique guide. The guide can be customized by the user to suit surgeon preferences for future procedures. The guide may also be used for pre-surgical training, continuing education, and for a sales representative to use as a teaching or sampling tool. Information provided by the guide may be provided via text, audio, images, or video. The example user interfaces of FIG. 15-18 illustrate a particular procedure and how the virtual tray system highlights different items for use in the procedure.

Referring to user interface 1500 of FIG. 15, the user has selected the guide tab 1502. Upon selecting tab 1502, the virtual tray system may, for example, retrieve a number of steps for a procedure to be performed that involves the actual tray. This procedure may be specified by the user, or the procedure may be associated with the actual tray. As shown in FIG. 15, the procedure starts at a “Step 1” in which an item “Guide-Wire” is identified. The item indicated in the step is then highlighted in the virtual tray (shown as 1504). Each step may identify other items to be used in the procedure (from the virtual tray or another tray not displayed, as shown below) and identifiers for the items to be used. The current step 1510 is shown highlighted. The user may be able to mark when a particular step is completed by, for example, touching the step on a touchscreen display.

Further, fields 1506, 1508 may be updated based on the instructions displayed. For example, for a set of instructions, fields 1506 are updated to show the total number of devices needed to follow the particular step, and the number of devices used or damaged among such devices. Further, in fields 1508, the user can mark other items as used or damaged as they are used while following the instructions.

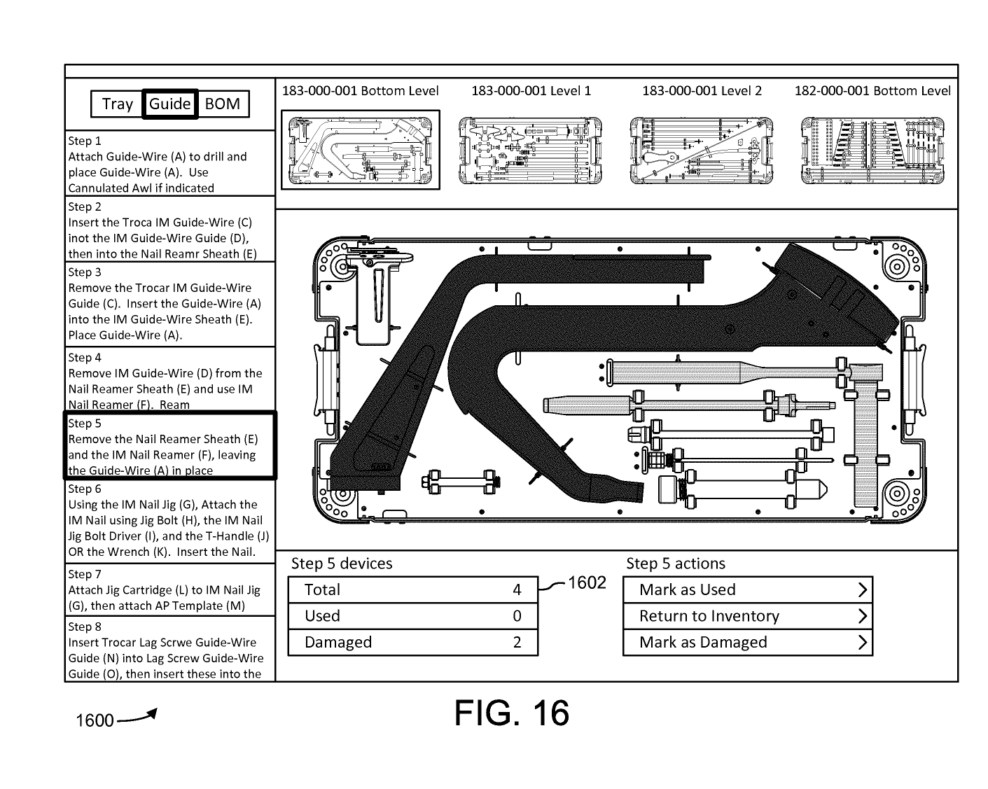

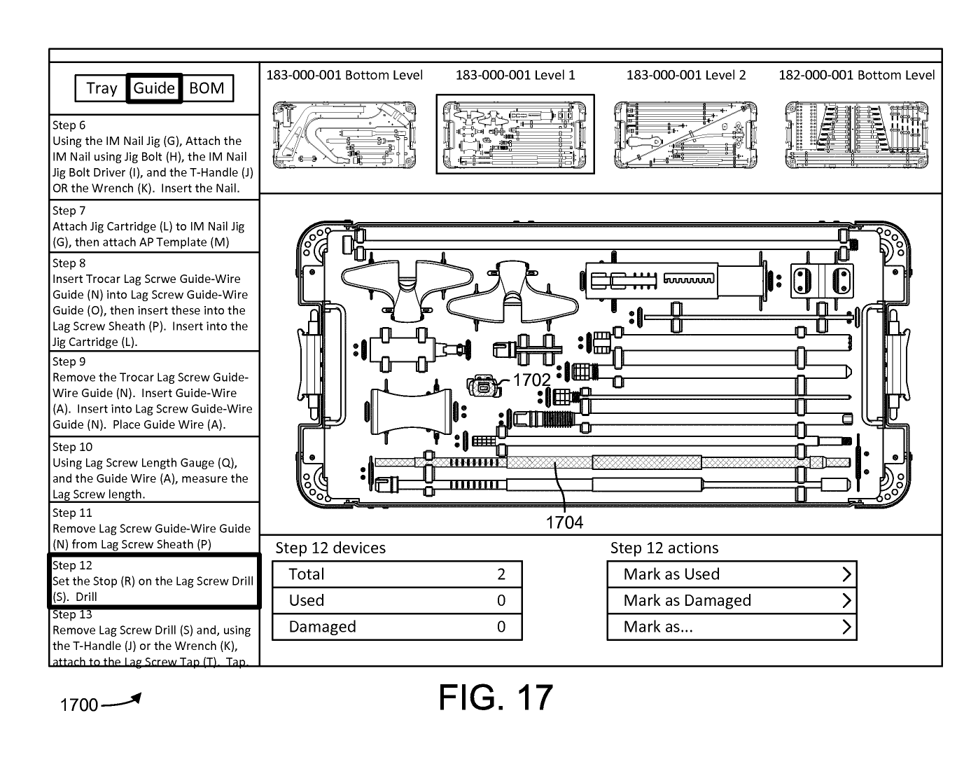

Referring to user interface 1600 of FIG. 16, the procedure has progressed from “Step 1” (FIG. 15) to “Step 5”. In this step, the user may need items from a different tray. User interface 1600 is shown to update the virtual tray display to show the tray with new items to be used in the procedure. As the virtual tray is updated, fields 1602 may be updated, to indicate how many of the items are to be used during the step, and if any of the items are used or damaged. Referring to user interface 1700 of FIG. 17, the procedure has progressed to “Step 12”, in which items 1702, 1704 from another tray are to be used.

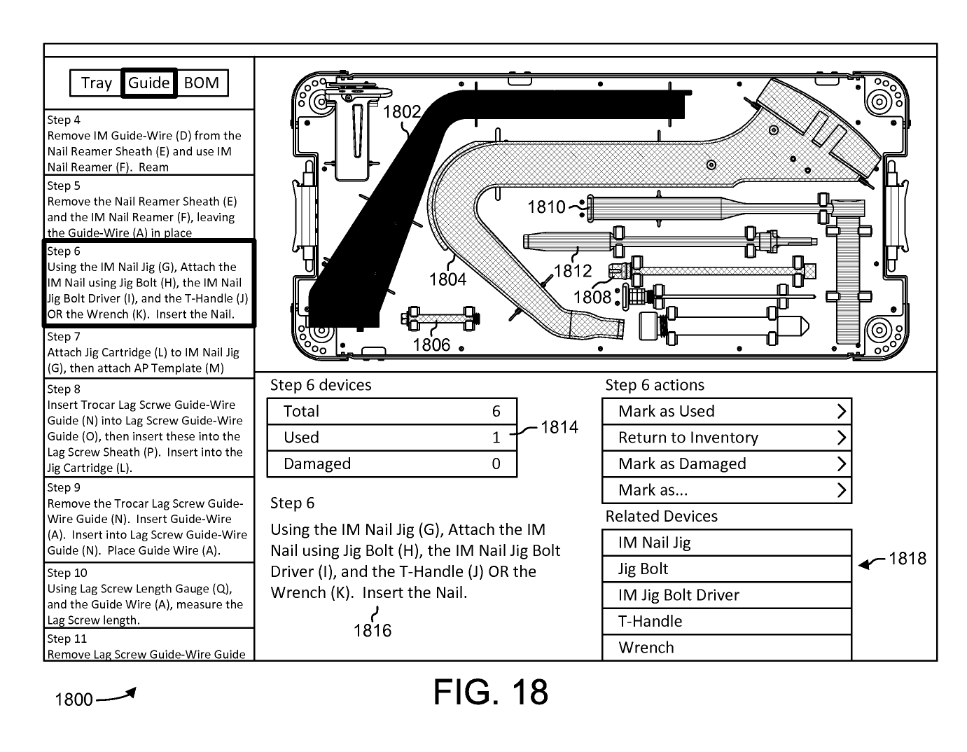

Referring now to user interface 1800 of FIG. 18, the user may be presented with further information for a given step. In the example of FIG. 18, the user is on or has selected “Step 6”, which requires the use of several items from a particular tray. In particular, several items 1802, 1804, 1806, 1808, 1810, 1812 are shown as marked in some form (e.g., the items may be used, damaged, or not yet used), indicating the items to be used to complete the step. One of the items 1802 is shown marked as used (also indicated by field 1814). The user may be presented with options for marking an item as used, returning the item to the inventory (e.g., placed back in the tray), marking an item as damaged, or otherwise. This marking may allow the user to indicate if the step was completed or partially completed successfully.

User interface 1800 is shown to further include fields 1816, 1818 for listing the step and listing the related devices for the step (e.g., the items to be used during the step). User interface 1800 may be configured to present such information upon selection of the step by a user, or may automatically present the information when the user indicates to the virtual tray system that he/she has reached the step.

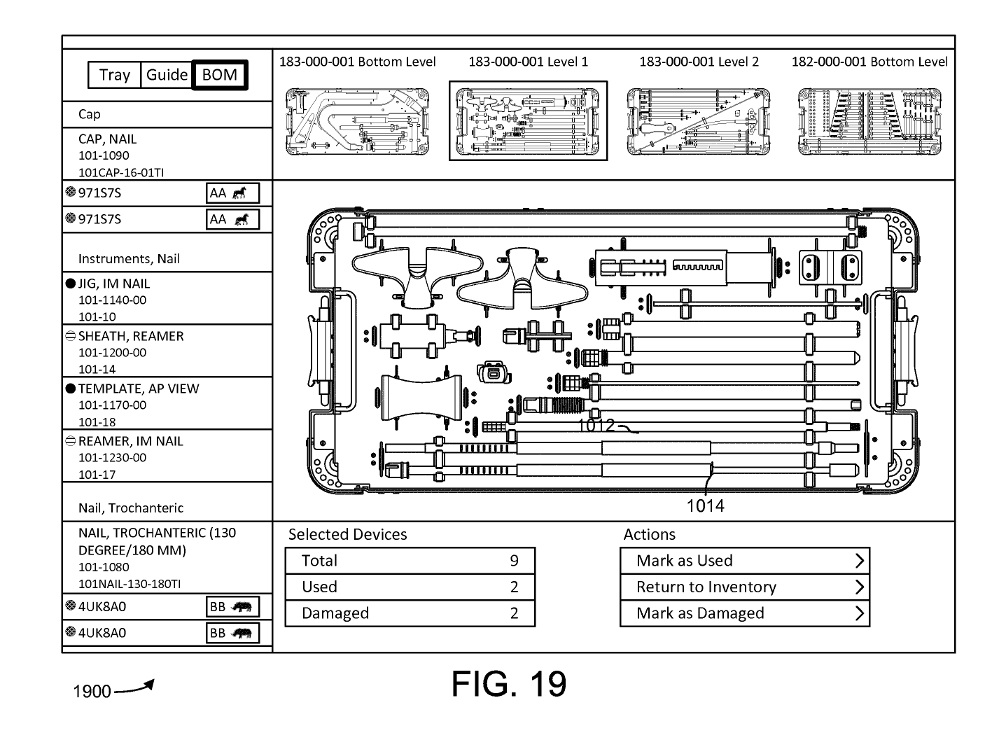

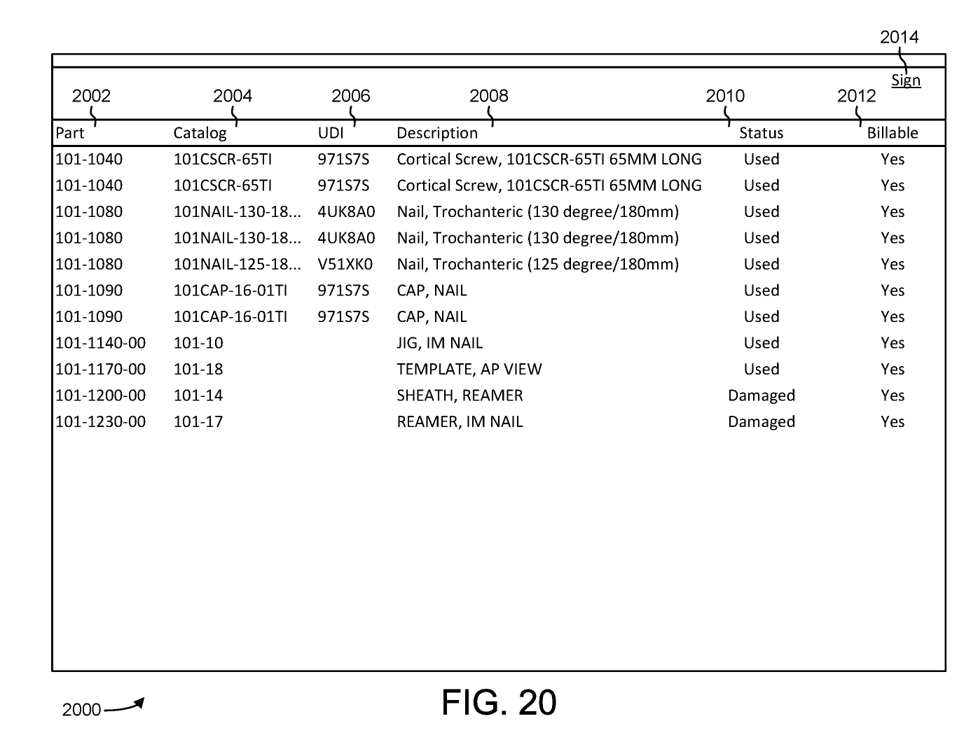

Referring generally to FIGS. 19-20, billing features of the virtual tray are described in greater detail. As described above, items from the tray that are used during a procedure are tracked. An invoice (e.g., BOM) may be generated by the virtual tray system based on the items used. The invoice may be generated at any time after an operation and may be generated based on the use of one or more particular items, based on a request for an invoice from medical personnel, or the like.

Referring to user interface 1900 of FIG. 19, the user has selected the bill of materials (BOM) tab 1902 to view items that have been used and/or damaged. In this example, the user may select to view the current BOM. Referring to user interface 2000 of FIG. 20, an example BOM is shown. For each item included in the BOM, a part number 2002, catalog number 2004, UDI 2006, description 2008, status 2010, and billable status 2012 is included. Part number 2002, catalog number 2004, and UDI 2006 may identify the item and type of item being billed. Status 2010 indicates whether the item has been used (or damaged), and billable status 2012 indicates whether the item can be billed for by the virtual tray system. The user may confirm the BOM by selecting the “Sign” option 2014 (or another similar option presentable to the user) that allows the user to electronically sign off on the purchase (or verify the purchase in any other way).

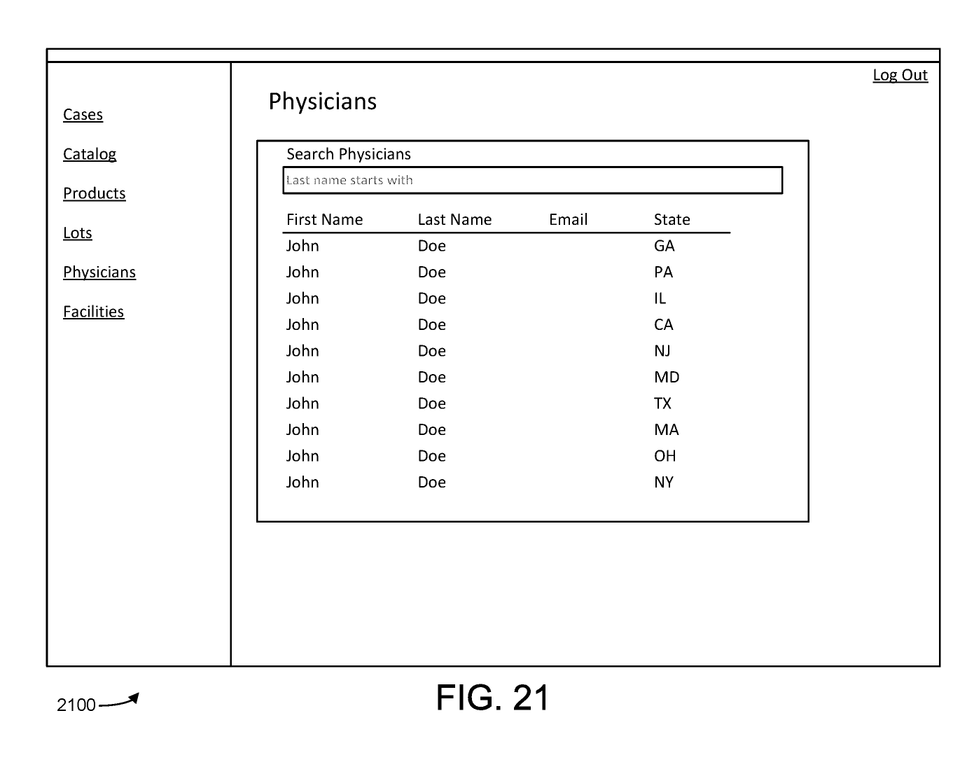

The virtual tray system in some embodiments provides an administrative console through which a user, can log into the system or can access information related to another user of the system. In one embodiment, the user may cause a user interface such as user interface 2100 of FIG. 21 to be presented on the screen. In user interface 2100, the user may browse a list of personnel (e.g., physicians in this example) to log in under that account or to access information about that user.

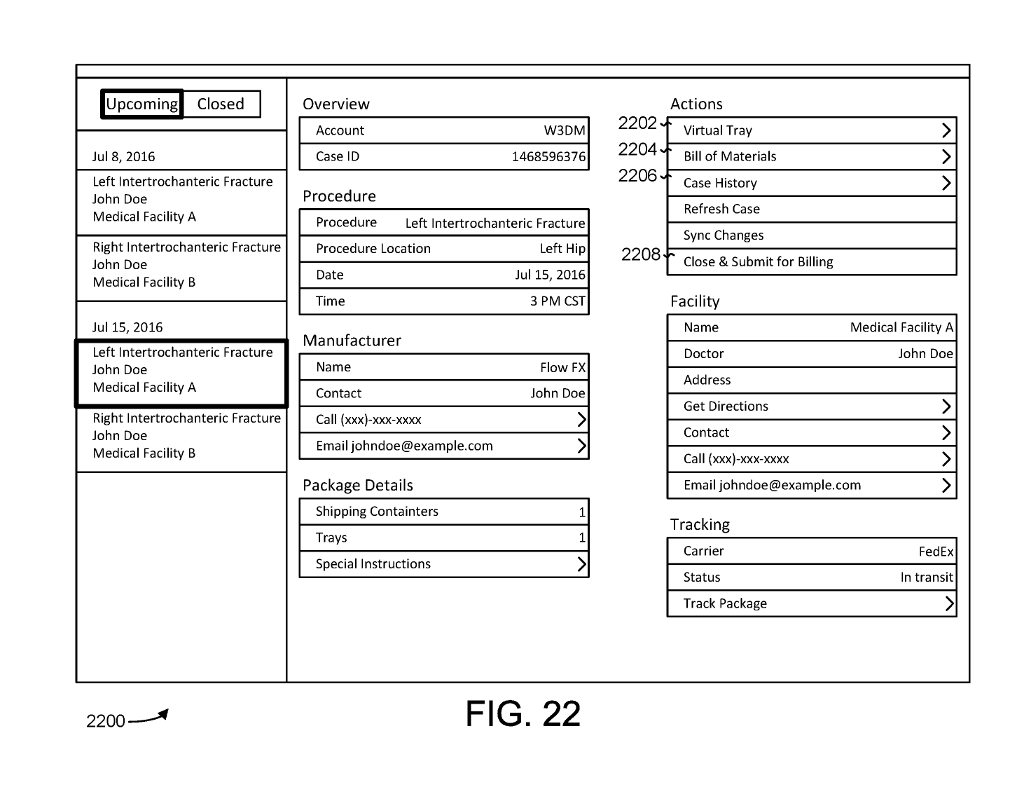

The user may further use the virtual tray system user interface to access other information. For example, the user may be able to browse a list of cases (described below in FIG. 22), a catalog or list of products available to the user and medical facility (e.g., products that can be provided via a tray in the future to the medical facility), or a list of other facilities, manufacturers, or distributors. The virtual tray system may allow access to user interface 2100 (and similar interfaces) based on a user login and/or user credentials.

Referring now to user interface 2200 of FIG. 22, case information for a particular case is shown in greater detail. A case may generally include a procedure to be performed on a patient, and a tray of items to be used in the procedure. In some embodiments, the virtual tray system may associate one or more items or trays with the procedure or the case. The user may be able to view case information via the virtual tray user interface, and more particularly information relating to the tray with the items to be used in the procedure. The user may be able to select between many upcoming cases, and to view completed or closed cases.

User interface 2200 may include an option 2202 to go to the virtual tray to be used, and an option 2204 to view a bill of materials for items used so far. The user may further select an option 2206 to view the case history (e.g., items and trays used in the past) or an option 2208 to close the case. In some embodiments, closing the case may generate an invoice for the personnel or medical facility.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. Other substitutions, modifications, changes, and omissions may be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, networked systems or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can include RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions. The machine-executable instructions may be executed on any type of computing device (e.g., computer, laptop, etc.) or may be embedded on any type of electronic device (e.g., a portable storage device such as a flash drive, etc.).

Although the figures may show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. A virtual tray system, comprising:
a tray inventory system configured to track a current inventory of a tray, the inventory including a plurality of items, and further configured to associate one or more details with each item in the tray;
a database configured to store the current inventory of the tray;
a user interface system configured to generate a visual representation of the inventory of the tray for display on a user interface of a user device; and
a communications system configured to receive an input relating to a change in status of at least one of the plurality of items in the tray;
wherein, based on the input:
the tray inventory system is configured to update the current inventory of the tray in the database; and
the user interface system is configured to update the visual representation of the tray based on the input.

2. The system of claim 1, wherein the plurality of items comprises medical devices, instruments, tools, implants, or a combination thereof, and wherein the tray and plurality of items are provided for use in a medical facility.

3. The system of claim 1, wherein the user interface system is configured to generate a visual representation of the tray for display on a touchscreen;
wherein the input is received via a touch on the touchscreen.

4. The system of claim 1, wherein a monitoring system remote from the virtual tray system is configured to generate the input, wherein the monitoring system comprises at least one of RFID readers, cameras, image recognition devices, or mobile devices.

5. The system of claim 4, wherein the monitoring system comprises a RFID reader and the item is associated with a RFID tag, and the monitoring system generates the input upon the detection of the RFID tag of the item when the item is removed from the tray.

6. The system of claim 1, wherein the input indicates one of:
the use of the item in a medical procedure;
the addition of an item to the tray;
the removal of an item from the tray;
the removal of an item from the tray due to damage of the item; or
feedback relating to the item.

7. The system of claim 6, wherein the feedback relating to the item is an indication that the item is damaged.

8. The system of claim 1, wherein the one or more details for each of the plurality of items comprises a unique device identifier (UDI); and
wherein the input comprises a unique device identifier (UDI) of an item and the tray inventory system is configured to update the status of an item in the database by matching the UDI with the stored UDI for the item in the database.

9. The system of claim 1, wherein the one or more details for an item comprises at least one of instructions for using the item in a medical procedure, a sterilization technique for the item, or a recommended use for the item;
wherein the tray inventory system is configured to retrieve the details for the item from one of the database or a remote system and the user interface system is configured to present the details on the user interface.

10. The system of claim 1, further comprising:
a billing system configured to generate an invoice relating to the usage of one or more items in the tray.

11. The system of claim 1, wherein the tray is a training tray configured for training personnel to familiarize personnel with the items in the virtual tray and to provide information relating to a procedure associated with the items.

12. The system of claim 1, further comprising:
a user identification system configured to determine an access level for one or more users for accessing the tray;
wherein the input comprises a user ID and the user identification system is configured to determine if the user is allowed to use an item based on matching an access level associated with the item with the access level associated with the user ID.

13. A method, comprising:
receiving from a manufacturer, by a virtual tray system, tray inventory information for a tray, wherein the inventory information comprises one or more details for a plurality of items in the tray;
storing, by the virtual tray system, the tray inventory information in a database;
receiving, by the virtual tray system, updated tray inventory information from a distributor that received the tray from the manufacturer and updating, by the virtual tray system, the tray inventory information in the database;
receiving, by the virtual tray system, updated tray inventory information from a medical facility that received the tray from the distributor and updating, by the virtual tray system, the tray inventory information in the database; and
receiving an input relating to a change in status of at least one of the plurality of items in the tray during a medical procedure and updating, by the virtual tray system, the tray inventory information in the database.

14. The method of claim 13, further comprising:
providing, by the virtual tray system for one of the manufacturer, distributor, health care institution, or a user device, a visual representation of the inventory of the tray;
wherein the virtual tray system is configured to update the visual representation of the inventory of the tray upon receiving updated tray inventory information.

15. The method of claim 13, wherein the updated tray inventory information comprises one of:
an indication of the use of an item in the tray in a medical procedure;
the addition of an item to the tray;
the removal of an item from the tray;
the removal of an item from the tray due to damage of the item; or feedback relating to the item.

16. The method of claim 13, wherein the updated tray inventory information comprises a unique device identifier (UDI) for an item; and
wherein updating the tray inventory information comprises comparing the UDI of the item with the UDI of the item stored in the database.

17. The method of claim 13, wherein the plurality of items comprises medical devices, tools, and implants, and wherein the tray and plurality of items are provided for use in a medical facility.

18. A method, comprising:
receiving, at a virtual tray system from a monitoring system, an indication of the use of an item carried in a tray;
updating, by the virtual tray system, an inventory of the tray based on the use of the item;
updating, by the virtual tray system, a database configured to store the current inventory of the tray; and
updating, by the virtual tray system, a visual representation of the tray for display on a user device.

19. The method of claim 18, further comprising:
generating, by the virtual tray system, a purchase order for the user of the item in the tray; and
providing, by the virtual tray system, an invoice comprising a plurality of purchase orders to the user device.

20. The method of claim 18, further comprising providing information associated with an item in the tray on the user device, wherein the information comprises at least one of:
instructions for using the item in a medical procedure;
a sterilization technique for the item; or
a recommended use for the item;
wherein the information provided is retrieved by the virtual tray system from one of the database or a remote system.
